(19) United States
(12) Patent Application Publication
(10) Pub. No.: US 2010/0168135 A1
(43) Pub. Date: Jul. 1, 2010

(54) Method of Inducing Cleavage of Amyloid Precursor Protein to Form a Novel Fragment

(76) Inventors: Kim Nicholas Green, Newport Beach, CA (US); Tilman Oltersdorf, Cardiff, CA (US); Eckard Weber, San Diego, CA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/638,219

(22) Filed: Dec. 15, 2009

Related U.S. Application Data
(60) Provisional application No. 61/122,689, filed on Dec. 15, 2008.
(60) Provisional application No. 61/122,694, filed on Dec. 15, 2008.
(60) Provisional application No. 61/122,704, filed on Dec. 15, 2008.
(60) Provisional application No. 61/122,705, filed on Dec. 15, 2008.

(51) Int. Cl. A61K 31/437; A61K 31/429; A61K 31/519; A61K 31/4745; C07K 2/00; C12Q 1/02

(52) U.S. Cl. 514/259.1; 514/278; 514/300; 514/368; 514/292; 530/300; 435/29

(57) ABSTRACT

The present invention provides a method of inducing cleavage of amyloid precursor protein to produce an approximately 17 kilodalton carboxy-terminal fragment of amyloid precursor protein in a subject, the method comprising administering a heterocyclic compound or a pharmaceutically acceptable salt, hydrate or prodrug thereof to a subject in need thereof, wherein the approximately 17 kilodalton fragment includes the carboxyterminal amino acid sequence of amyloid precursor protein and amyloid-beta amino acid sequence. Also provided is a screening method for identifying compounds induce cleavage of amyloid precursor protein to produce the approximately 17 kilodalton carboxy-terminal fragment of amyloid precursor protein.

The present application claims priority benefit of U.S. Provisional Application Nos. 61/122,689, 61/122,694, 61/122,704 and 61/122,705, each of which was filed on Dec. 15, 2008, and each of which is incorporated by reference in its entirety.

### FIELD OF THE INVENTION

This invention relates to the regulation of the processing of amyloid precursor protein (APP), and particularly to inducing cleavage of APP to form a novel protein fragment.

### BACKGROUND OF THE INVENTION

Alzheimer's Disease (AD) is a neurodegenerative disorder for which there are only symptomatic treatments, with limited efficacy. Certain amyloid-beta (Aβ) fragments of APP, notably Aβ₁₋₄₀ and Aβ₁₋₄₂ have been implicated in the pathology of AD. Reduction of Aβ has been pursued as an approach to modify the course of AD (Barten, D. and C. Albright, Mol. Neurobiol. 37: 171-186 (1998)). However, to date, no approved therapies have resulted from this approach.

Attempts have been made to treat AD with both active and passive immunization against Aβ. One such immunization approach has already failed in human testing (Holmes, C. et al., Lancet 372: 216-23 (2008)). A limitation of Aβ immunotherapy may be that it targets only Aβ that is already formed. It does not slow or halt production of new Aβ, and in fact, may even encourage increased production of new Aβ.

Other attempts to treat AD have involved interrupting known enzymes from the processing of APP before deleterious Aβ fragments can be produced. These enzyme targets are gamma-secretase and beta-secretase. Gamma-secretase inhibitors have not proved useful, because many such inhibitors affect cleavage of other gamma-secretase substrates and as a result can be toxic (Czirr, E. and S. Weggen, Neurodegenerative Dis. 3: 298-302 (2006); Tomita, T., Nauyn-Schmiedegerg's Arch. Pharmacol. 377: 295-300 (2008)); Milano, J. et al., Toxicological Sciences 82: 341-358 (2004)).

Gamma-secretase modulators also have not proved useful. Examples of gamma secretase modulators include non-steroidal anti-inflammatory drugs (NSAIDs), which are allosteric modulators of gamma secretase. Such compounds are not toxic, but compounds that have entered clinical testing have only high micromolar in vitro potency, such that they are too weak to have sufficient clinical effects (Czirr, E. and S. Weggen, Neurodegenerative Dis. 3: 298-304 (2006)). The prototype gamma secretase modulator, Flurizan, recently failed a Phase III clinical trial.

In addition, prior attempts at treating AD with beta-secretase inhibitors have not proven useful, because the large binding pocket of beta-secretase combined with its membrane location creates a challenge to design inhibitors that cross the blood-brain barrier in sufficient concentration to be useful (Barten, D. and C. Albright, Mol. Neurobiol. 37: 171-186 (1998); John, V., Curr. Top. Med. Chem. 6: 569-78 (2006); Venugopal, C. et al., CNS & Neurological Disorders—Drug Targets 7: 278-294 (2008)).

Thus, there remains a need in the art for an effective treatment of AD.

### BRIEF SUMMARY OF THE INVENTION

The present invention provides a method of inducing cleavage of APP to produce an approximately 17 kilodalton (kDa) carboxy-terminal fragment of APP in a subject, the method comprising administering a heterocyclic compound having the general Formula (I):

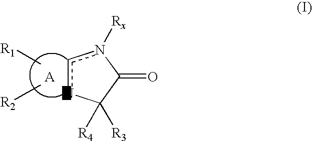

or a pharmaceutically acceptable salt, hydrate or prodrug thereof to a subject in need thereof, wherein the approximately 17 kDA fragment includes the carboxy-terminal amino acid sequence of APP and amyloid-beta amino acid sequence, and wherein each of Rₓ, R₁, R₂, R₃, R₄ are as defined herein.

The present invention also provides an approximately 17 kDa APP fragment that includes the carboxy-terminal amino acid sequence of APP and amyloid-beta amino acid sequence.

The present invention also provides a method for screening for a compound that cleaves APP to generate an approximately 17 kDa fragment of APP, the method comprising: (a) exposing cells that produce APP or fragments thereof to a test compound, and (b) detecting the amount of the approximately 17 kDa fragment, wherein the approximately 17 kDa fragment includes the carboxy-terminal amino acid sequence of APP and amyloid-beta amino acid sequence, and wherein an increase in the amount of the approximately 17 kDa fragment in cells that are exposed to the compound, relative to the amount of the approximately 17 kDa fragment in cells that are not exposed to the compound, indicates that the compound cleaves APP to generate the approximately 17 kDa fragment.

The present invention provides a method of inducing cleavage of APP to produce an approximately 17 kDa carboxy-terminal fragment of APP in a subject, the method comprising administering a compound that is not a compound having the general Formula (I):

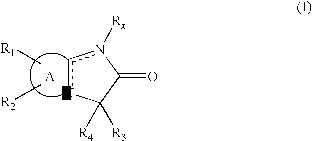

or a pharmaceutically acceptable salt, hydrate or prodrug thereof, wherein each of Rₓ, R₁, R₂, R₃, R₄ are as defined herein.

### BRIEF DESCRIPTION OF THE DRAWINGS/FIGURES

FIGS. 1A and 1B are bar graphs that depict the effect of the compound ST101 on Aβ production by N2a cells. FIG. 1A is a bar graph that depicts the Aβ concentration in the cell culture medium as a function of ST101 concentration compared to control after 24 hours of treatment. FIG. 1B is a bar graph that depicts the ratio of Aβ 1-42 to Aβ 1-40 as a function of ST101 concentration compared to control.

FIGS. 2A, 2B and 2C are graphs that depict the effect of ST101 in 3xTg-AD mice in the Morris water maze. FIG. 2A is a line graph depicting latency (in seconds) during training over a period of seven days, compared to control mice. FIGS. 2B and 2C are bar graphs that depict latency (in seconds) and number of crosses over the platform location at 24 and 72 hours after training in ST101-treated animals and control mice.

FIGS. 3A and 3B are bar graphs that depict the effect of ST101 on Aβ in brain tissue from 3xTg-AD mice. FIG. 3A depicts the amounts of soluble Aβ₁₋₄₀ and Aβ₁₋₄₂ in brain tissue in mice treated with ST101, relative to control mice. FIG. 3B a bar graph that depicts the amounts of insoluble Aβ₁₋₄₀ and Aβ₁₋₄₂ (formic acid extraction) in mice treated with ST101, relative to control mice.

FIG. 4 is a Western blot that depicts APP carboxy-terminal fragments detected by antibody CT20 in the brains of ST101-treated (S) 3xTg-AD mice, relative to untreated (C) 3xTg-AD mice.

FIG. 5 is a Western blot that depicts APP and degradation fragments detected by antibody CT20 in the brains of ST101-treated (S) 3xTg-AD mice, relative to untreated (C) 3xTg-AD mice. * indicates full length APP species, ** indicates major degradation products, and “Actin” stands for anti-beta-actin antibody as a protein loading control.

FIG. 6 is a drawing that depicts a proposed amyloid processing pathway leading to a novel APP carboxy-terminal fragment.

FIGS. 7A-C are Western blots that depict APP C-terminal fragments detected by antibody CT20 in the brains of ST101-treated (S) 3xTg-AD mice, relative to untreated (C) 3xTg-AD mice.

FIG. 8 is a Western blot that depicts C-terminal fragments detected by antibody 1565-1 (Epitomics Inc.) in the brains of ST101-treated (S) 3xTg-AD mice, relative to untreated (C) 3xTg mice-AD.

### DETAILED DESCRIPTION OF THE INVENTION

Unless defined otherwise, all technical and scientific terms used herein have the same meaning as is commonly understood by one of ordinary skill in the art to which this invention belongs.

The present invention provides a method of inducing cleavage of APP to produce an approximately 17 kDa carboxy-terminal fragment of APP in a subject, the method comprising administering a heterocyclic compound having the general Formula (I):

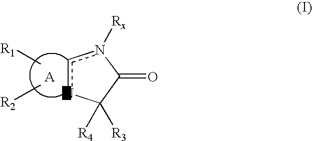

or a pharmaceutically acceptable salt, hydrate or prodrug thereof to a subject in need thereof, wherein the approximately 17 kDa fragment includes the carboxyterminal amino acid sequence of APP and amyloid-beta amino acid sequence, and wherein each of Rₓ, R₁, R₂, R₃, R₄ are as defined herein.

In one embodiment, administering a heterocyclic compound having the general Formula (I) results in a decrease in the production of one or more of Aβ₁₋₄₂, Aβ₁₋₄₀, the C99 fragment of APP, and/or the C83 fragment of APP.

In another embodiment, the subject to whom a heterocyclic compound having the general Formula (I) is administered has AD. In another embodiment, the subject is or has been diagnosed with AD. In another embodiment, the subject has mild cognitive impairment. In another embodiment, the subject is or has been diagnosed with mild cognitive impairment.

In another embodiment, the AD is treated. In another embodiment, the mild cognitive impairment is treated. As used herein, “treatment” means any manner in which the symptoms of a condition, disorder or disease are ameliorated or otherwise beneficially altered.

In one embodiment, the AD is prevented. In another embodiment, the mild cognitive impairment is prevented. “Preventing” AD or cognitive impairment, as used herein, refers to preventing the occurrence of one or more symptoms of AD in a subject.

As used herein, amelioration of the symptoms of a particular disorder by administration of a particular pharmaceutical composition refers to any lessening, whether permanent or temporary, lasting or transient, that can be attributed to or associated with administration of the composition.

In another embodiment, the subject is or has been screened to determine whether the subject has AD. The screening can be performed by examining the subject. Alternatively, the screening can be performed by assaying one or more biological markers of AD.

In another embodiment, the subject has been diagnosed as predisposed to AD. In another embodiment, the subject is screened or has been screened to determine whether the subject is predisposed to develop AD. The screening can be performed by examining the subject. Alternatively, the screening can be performed by assaying one or more biological markers of predisposition to AD.

In another embodiment, the subject is a human subject.

The present invention also provides an isolated approximately 17 kDa APP fragment that includes the carboxy-terminal amino acid sequence of APP and amyloid-beta amino acid sequence.

The present invention also provides a composition comprising the approximately 17 kDA fragment of the invention. In another embodiment, the composition also comprises cell culture lysate and/or medium.

The present invention also provides a container comprising the approximately 17 kDA fragment of the invention. In another embodiment, the container is a microtube. In another embodiment, the container is a test tube. In another embodiment, the container is pipette or a micropipette. In another embodiment, the container is a microarray apparatus. In another embodiment, the container is a microtiter plate. In another embodiment, the container is a component of a screening assay apparatus.

The present invention also provides a method for screening for a compound that cleaves APP to generate an approximately 17 kDa fragment of APP, the method comprising: (a) exposing cells that produce APP or fragments thereof to a test compound, and (b) detecting the amount of the approximately 17 kDa fragment, wherein the approximately 17 kDa fragment includes the carboxy-terminal amino acid sequence of APP and amyloid-beta amino acid sequence, and wherein an increase in the amount of the approximately 17 kDa fragment of cells exposed to the compound, relative to the amount of the approximately 17 kDa fragment in cells that are not exposed to the compound, indicates that the compound induces cleavage of APP to generate the approximately 17 kDa fragment.

Alternatively, one can detect the presence of the free amino-terminus of the approximately 17 kDa fragment, or one can detect the free carboxy-terminus of APP generated by the cleavage that created the 17 kDa fragment.

The present invention also provides a method for screening for a compound that cleaves APP to generate an approximately 17 kDa fragment of APP, the method comprising: (a) exposing cells that produce APP or fragments thereof to a test compound, and (b) detecting the approximately 17 kDa fragment, wherein the approximately 17 kDa fragment includes the carboxy-terminal amino acid sequence of APP and amyloid-beta amino acid sequence, and wherein the presence of the approximately 17 kDa fragment of cells exposed to the compound, relative to the absence of the approximately 17 kDa fragment in cells that are not exposed to the compound, indicates that the compound induces cleavage of APP to generate the approximately 17 kDa fragment.

In one embodiment, a screening method of the present invention further comprises (c) determining whether the amount of one or more of Aβ₁₋₄₂, Aβ₁₋₄₀, the C99 fragment of APP, or the C83 fragment of APP in cells exposed to the compound is decreased, relative to the amount of Aβ₁₋₄₂, Aβ₁₋₄₀, the C99 fragment of APP, or the C83 fragment of APP in cells that are not exposed to the compound.

In one embodiment, a screening method of the present invention is carried out in vivo.

In another embodiment, a screening method of the present invention is carried out in vitro. In this embodiment, either the presence of or the amount of the approximately 17 kDa fragment in the cell culture can be measured, for cells that are exposed to the compound and for control cells that are not exposed to the compound. For example, an increase in the amount of the approximately 17 kDa fragment in the cell culture of cells exposed to the compound, relative to the amount of the approximately 17 kDa fragment in the cell culture of cells that are not exposed to the compound, indicates that the compound cleaves APP to generate the approximately 17 kDa fragment. Alternatively, the presence of the approximately 17 kDa fragment in the cell culture of cells exposed to the compound, relative to the absence of the approximately 17 kDa fragment in the cell culture of cells that are not exposed to the compound, indicates that the compound cleaves APP to generate the approximately 17 kDa fragment.

In another embodiment, a screening method of the present invention is carried out in cells in culture.

In another embodiment, a screening method of the present invention further comprises determining whether the amount of one or more of Aβ₁₋₄₂, Aβ₁₋₄₀, the C99 fragment of APP, or the C83 fragment of APP in the cell culture lysate of cells exposed to the compound is decreased, relative to the amount of Aβ₁₋₄₂, Aβ₁₋₄₀, the C99 fragment of APP, or the C83 fragment of APP in the cell culture medium of cells that are not exposed to the compound.

In another embodiment, a screening method of the present invention is carried out in a high-throughput manner. In another embodiment, a screening method of the present invention is automated. In another embodiment, a screening method of the present invention is computer-controlled.

In another embodiment, a plurality of cultured cells are exposed separately to a plurality of test compounds, e.g. in separate wells of a microtiter plate. In this embodiment, a large number of test compounds may be screened at the same time.

The test compounds may be presented to the cells or cell lines dissolved in a solvent. Examples of solvents include, DMSO, water and/or buffers. DMSO may be used in an amount below 2%. Alternatively, DMSO may be used in an amount of 1% or below. At this concentration, DMSO functions as a solubilizer for the test compounds and not as a permeabilization agent. The amount of solvent tolerated by the cells must be checked initially by measuring cell viability with the different amounts of solvent alone to ensure that the amount of solvent has no effect on the cellular properties being measured.

Suitable buffers include cellular growth media, for example Iscove's media (Invitrogen Corporation) with or without 10% fetal bovine serum. Other known cellular incubation buffers include phosphate, PIPES or HEPES buffers. One of ordinary skill in the art can identify other suitable buffers with no more than routine experimentation.

Cells that produce APP or fragments thereof include, but are not limited to IMR-32, BV-2, T98G, NT2N and N2A cells. In another embodiment, the cells are N2A cells.

In another embodiment, the cells that produce APP or fragments thereof include cells into which nucleic acid encoding APP or mutated APP has been introduced, e.g., by transfection.

The approximately 17 kDa APP fragment, Aβ₁₋₄₂, Aβ₁₋₄₀, the C99 fragment of APP, and/or the C83 fragment of APP can also be detected, for example, using gel electrophoresis. The 17 kDa APP fragment, Aβ₁₋₄₂, Aβ₁₋₄₀, the C99 fragment of APP, and/or the C83 fragment of APP can also be detected using a sandwich ELISA assay employing a first monoclonal antibody directed, e.g., against the N-terminus of the 17 kDa fragment and a second monoclonal antibody directed, e.g., against another region of the 17 kDa fragment, e.g., the carboxy-terminus of the 17 kDa fragment.

The approximately 17 kDa APP fragment, Aβ₁₋₄₂, Aβ₁₋₄₀, the C99 fragment of APP, and/or the C83 fragment of APP can also be detected, for example, using mass spectrometry, with or without prior immunoprecipitation by an antibody.

In another embodiment, the approximately 17 kDa fragment is isolated.

The term “isolated” as used herein means separated from the brain of a subject. In another embodiment, the approximately 17 kDa fragment is present in an electrophoretic gel. In another embodiment, the approximately 17 kDa fragment is present in cell culture lysate or medium.

The “approximately 17 kDa fragment” of APP is the fragment of APP that contains the C-terminal sequence of APP and the amyloid-beta sequence of APP. The approximately 17 kDa fragment is not the C99 fragment of APP or the C83 fragment of APP.

The present invention also provides a method of inducing cleavage of APP to produce an approximately 17 kDa carboxy-terminal fragment of APP in a subject, the method comprising administering a compound that is not a compound having the general Formula (I):

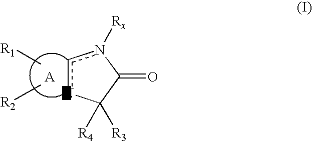

or a pharmaceutically acceptable salt, hydrate or prodrug thereof, wherein each of Rₓ, R₁, R₂, R₃, R₄ are as defined herein. In one embodiment, the compound is not a compound disclosed in any of U.S. application Ser. No. 11/872,408 (published as US 2008/0103157 A1); U.S. application Ser. No. 11/872,418 (published as US 2008/0103158 A1); U.S. Pat. No. 6,635,652; U.S. Pat. No. 7,141,579; and international Appl. No. PCT/JP2007/070962 (published as WO 2008/047951), each of which is incorporated by reference in its entirety. In another embodiment, the compound is not spiro(imidazo[1,2-a]pyridin-2(3H)-one-3,2′-indan).

In another embodiment, administering a compound that is not a compound having the general Formula (I) results in a decrease in the production of one or more of Aβ₁₋₄₂, Aβ₁₋₄₀, the C99 fragment of APP, and/or the C83 fragment of APP.

In another embodiment, the subject to whom a heterocyclic compound having the general Formula (I) is administered has AD. In another embodiment, the subject is or has been diagnosed with AD. In another embodiment, the subject has mild cognitive impairment. In another embodiment, the subject is or has been diagnosed with mild cognitive impairment.

In another embodiment, the AD is treated. In another embodiment, the mild cognitive impairment is treated. As used herein, “treatment” means any manner in which the symptoms of a condition, disorder or disease are ameliorated or otherwise beneficially altered.

In one embodiment, the AD is prevented. In another embodiment, the mild cognitive impairment is prevented. “Preventing” AD or cognitive impairment, as used herein, refers to preventing the occurrence of one or more symptoms of AD in a subject.

As used herein, amelioration of the symptoms of a particular disorder by administration of a particular pharmaceutical composition refers to any lessening, whether permanent or temporary, lasting or transient, that can be attributed to or associated with administration of the composition.

In another embodiment, the subject is or has been screened to determine whether the subject has AD. The screening can be performed by examining the subject. Alternatively, the screening can be performed by assaying one or more biological markers of AD.

In another embodiment, the subject has been diagnosed as predisposed to AD. In another embodiment, the subject is or has been screened or has been screened to determine whether the subject is predisposed to develop AD. The screening can be performed by examining the subject. Alternatively, the screening can be performed by assaying one or more biological markers of predisposition to AD.

In another embodiment, the subject is a human subject.

The heterocyclic compound of the present invention can be administered at an effective oral dosage of 0.0005 mg per kilogram of body weight or higher. In one embodiment, the compound is administered as an active ingredient as part of a unit dosage form containing 5, 10, 15, 20, 25, 30, 35, 40, 45, 50, 55, 60, 65, 70, 75, 80, 85, 90, 95, or 100 mg of the compound.

Compositions for use in this invention include all compositions wherein the active ingredient is contained in an amount which is effective to achieve its intended purpose. While individual needs vary, determination of optimal ranges of effective amounts of each component is within the skill of the art. Typically, the active ingredient may be administered to mammals, e.g. humans, orally at a dose of 0.001 to 3 mg/kg, or an equivalent amount of the pharmaceutically acceptable salt thereof, per day of the body weight of the mammal being treated for AD. The active ingredient may be administered to mammals, e.g. humans, intravenously or intramuscularly at a dose of 0.001 to 3 mg/kg, or an equivalent amount of the pharmaceutically acceptable salt thereof, per day of the body weight of the mammal being treated for AD. Approximately 0.001 to approximately 3 mg/kg can be orally administered to treat or prevent such disorders. If another agent is also administered, it can be administered in an amount which is effective to achieve its intended purpose.

The unit oral dose may comprise from approximately 0.001 to approximately 200 mg, or approximately 0.5 to approximately 180 mg of the composition of the invention. The unit dose may be administered one or more times daily as one or more tablets, each containing from approximately 0.1 to approximately 90 mg, conveniently approximately 10 to 180 mg of the composition or its solvates. In one embodiment, the unit oral dose can be 10, 20, 30, 40, 50, 60, 70, 80, 90, 100, 110, 120, 130, 140, 150, 160, 170, or 180 mg of the compound.

In a topical formulation, the active ingredient may be present at a concentration of approximately 0.01 to 100 mg per gram of carrier.

In addition to administering the active ingredient as a raw chemical, the active ingredient may be administered as part of a pharmaceutical preparation containing suitable pharmaceutically acceptable carriers comprising excipients and auxiliaries, which facilitate processing of the active ingredient into preparations that can be used pharmaceutically. The preparations, particularly those preparations, which can be administered orally, such as tablets, dragees, and capsules, and also preparations, which can be administered rectally, such as suppositories, as well as suitable solutions for administration by injection or orally, can contain from approximately 0.01 to 99 percent, or from approximately 0.25 to 75 percent of active ingredient, together with the excipient.

The heterocyclic compound of Formula (I) can be in the form of hydrate or acid addition salts as a pharmaceutically acceptable salt. Possible acid addition salts include inorganic acid salts such as the hydrochloride, sulfate, hydrobromide, nitrate, and phosphate salts and organic acid salts such as acetate, oxalate, propionate, glycolate, lactate, pyruvate, malonate, succinate, maleate, fumarate, malate, tartrate, citrate, benzoate, cinnamate, methanesulfonate, benzenesulfonate, p-toluenesulfonate, and salicylate salts.

Acid addition salts are formed by mixing a solution of the particular compound of the present invention with a solution of a pharmaceutically acceptable non-toxic acid, such as hydrochloric acid, hydrobromic acid, fumaric acid, maleic acid, succinic acid, acetic acid, citric acid, lactic acid, tartaric acid, carbonic acid, phosphoric acid, sulfuric acid, oxalic acid, and the like. Basic salts are formed by mixing a solution of the particular compound of the present invention with a solution of a pharmaceutically acceptable non-toxic base, such as sodium hydroxide, potassium hydroxide, choline hydroxide, sodium carbonate, Tris, N-methyl-glucamine and the like.

The pharmaceutical compositions of the invention may be administered to any animal, which may experience the beneficial effects of the active ingredient. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner, which is itself known, e.g., by means of conventional mixing, granulating, dragee-making, dissolving, or lyophilizing processes. Thus, pharmaceutical preparations for oral use can be obtained by combining the active ingredient with solid excipients, optionally grinding the resultant mixture and processing the mixture of granules, after adding suitable auxiliaries, if desired or necessary, to obtain tablets or dragee cores.

Suitable excipients are, in particular: fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, using, e.g. maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, e.g. silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropymethyl-cellulose phthalate, are used. Dye stuffs or pigments may be added to the tablets or dragee coatings, e.g., for identification or in order to characterize combinations of active ingredient doses.

Other pharmaceutical preparations, which can be used orally, include push-fit capsules made of gelatin, as well as soft, sealed capsules made of gelatin and a plasticizer, such as glycerol or sorbitol. The push-fit capsules can contain the active ingredient in the form of granules, which may be mixed with fillers, such as lactose, binders such as starches, and/or lubricants such as talc or magnesium stearate and, optionally, stabilizers. In soft capsules, the active ingredient can be dissolved or suspended in suitable liquids, such as fatty oils, or liquid paraffin. In addition, stabilizers may be added.

Possible pharmaceutical preparations, which can be used rectally include, e.g. suppositories, which consist of a combination of one or more of the active ingredient with a suppository base. Suitable suppository bases are, e.g. natural or synthetic triglycerides, or paraffin hydrocarbons. In addition, it is also possible to use gelatin rectal capsules, which consist of a combination of the active ingredient with a base. Possible base materials include, e.g. liquid triglycerides, polyethylene glycols, or paraffin hydrocarbons.

Suitable formulations for parenteral administration include aqueous solutions of the active ingredient in water-soluble form, e.g. water-soluble salts and alkaline solutions. In addition, suspensions of the active ingredient as appropriate oily injection suspensions may be administered. Suitable lipophilic solvents or vehicles include fatty oils, e.g. sesame oil; or synthetic fatty acid esters, e.g. ethyl oleate or triglycerides or polyethylene glycol-400. Aqueous injection suspensions may contain substances, which increase the viscosity of the suspension include, e.g. sodium carboxymethyl cellulose, sorbitol, and/or dextran. Optionally, the suspension may also contain stabilizers.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady, Medicinal Chemistry: A Biochemical Approach, Oxford University Press, New York, pages 388 392 (1985)).

Also included within the scope of the present invention are dosage forms of the active ingredient, in which the oral pharmaceutical preparations comprise an enteric coating. The term “enteric coating” is used herein to refer to any coating over an oral pharmaceutical dosage form that inhibits dissolution of the active ingredient in acidic media, but dissolves rapidly in neutral to alkaline media and has good stability to long-term storage. Alternatively, the dosage form having an enteric coating may also comprise a water soluble separating layer between the enteric coating and the core.

The core of the enterically coated dosage form comprises an active ingredient. Optionally, the core also comprises pharmaceutical additives and/or excipients. The separating layer may be a water soluble inert active ingredient or polymer for film coating applications. The separating layer is applied over the core by any conventional coating technique known to one of ordinary skill in the art. Examples of separating layers include, but are not limited to sugars, polyethylene glycol, polyvinylpyrrolidone, polyvinyl alcohol, hydroxypropyl cellulose, polyvinyl acetal diethylaminoacetate and hydroxypropyl methylcellulose. The enteric coating is applied over the separating layer by any conventional coating technique. Examples of enteric coatings include, but are not limited to cellulose acetate phthalate, hydroxypropyl methylcellulose phthalate, polyvinyl acetate phthalate, carboxymethylethylcellulose, copolymers of methacrylic acid and methacrylic acid methyl esters, such as Eudragit®L 12.5 or Eudragit®L 100 (Rohm Pharma), water based dispersions such as Aquateric® (FMC Corporation), Eudragit®L 100-55 (Rohm Pharma) and Coating CE 5142 (BASF), and those containing water soluble plasticizers such as Citroflex® (Pfizer). The final dosage form is either an enteric coated tablet, capsule or pellet.

Examples of prodrugs of the compounds of the invention include the simple esters of carboxylic acid containing compounds (e.g. those obtained by condensation with a C₁₋₄ alcohol according to methods known in the art); esters of hydroxy containing compounds (e.g. those obtained by condensation with a C₁₋₄ carboxylic acid, C₃₋₆ dioic acid or anhydride thereof (e.g. succinic and fumaric anhydrides according to methods known in the art); imines of amino containing compounds (e.g. those obtained by condensation with a C₁₋₄ aldehyde or ketone according to methods known in the art); and acetals and ketals of alcohol containing compounds (e.g. those obtained by condensation with chloromethyl methyl ether or chloromethyl ethyl ether according to methods known in the art).

Symptoms of AD include confusion, disturbances in short-term memory, problems with attention, problems with spatial orientation, personality changes, language difficulties and mood swings. It is understood that the list of symptoms of AD may be expanded upon in the future as medical science continues to evolve. Thus, the term “symptoms of AD” is not to be limited to the list of symptoms provided herein.

As used herein an effective amount of a compound for treating a particular disease is an amount that is sufficient to ameliorate, or in some manner reduce, the symptoms associated with the disease. Such amount may be administered as a single dosage or may be administered according to a regimen, whereby it is effective. The amount may cure the disease but, typically, is administered in order to ameliorate the disease. Typically, repeated administration is required to achieve the desired amelioration of symptoms.

In the general Formula (I), the structural unit having the general Formula (II) may be one or more structural units selected from multiple types of structural units having the general Formula (III).

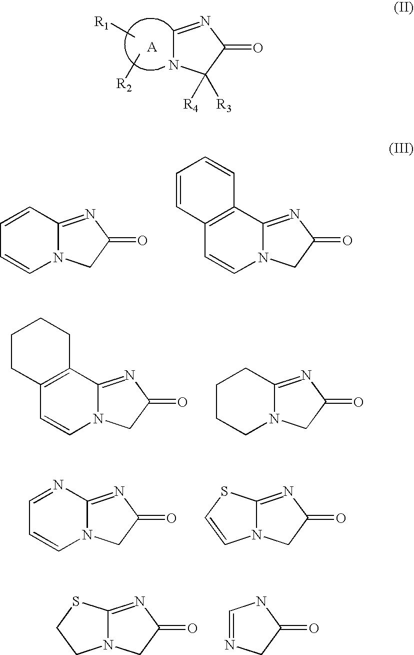

In the general Formula (I), Rₓ is methyl or nil. In the general Formula (I) and Formula (II), R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅ (wherein R₅ is phenyl (which may be substituted with C₁-C₆ alkyl, halogen atom or cyano) or thienyl) and —O—(CH₂)ₙ—R₆, wherein R₆ is a vinyl group, C₃-C₈ cycloalkyl group, or phenyl group, and n is 0 or 1.

In the general Formula (I) and Formula (II), R₃ and R₄ each are one or more functional groups independently selected from the group consisting of a hydrogen atom, C₁-C₆ alkyl group, C₂-C₆ alkenyl, C₃-C₈ cycloalkyl group, CH₂—R₅ (wherein R₅ is phenyl (which may be substituted with C₁-C₆ alkyl, halogen atom or cyano); naphtyl or thienyl) and —CH(R₈)—R₇. Alternatively, R₃ and R₄ together form a Spiro ring having the general Formula (IV):

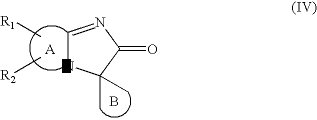

R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group. The above R₈ is a hydrogen atom or C₁-C₆ alkyl group.

Furthermore, in the general Formula (IV), the structural unit B may be one or more structural units selected from multiple types of structural units having the general Formula (V). The structural unit B binds at a position marked by * in the general Formula (V) to form a spiro ring, wherein R₉ is one or more functional groups selected from the group consisting of a hydrogen atom, halogen atom, hydroxy group, C₁-C₆ alkoxy group, cyano group, and trifluoromethyl group.

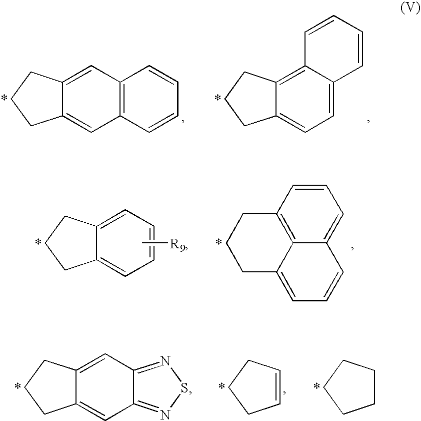

When the heterocyclic compound having the general Formula (I) has asymmetric carbon atoms in the structure, its isomer from asymmetric carbon atoms and their mixture (racemic modification) is present. In such cases, all of them are included in the heterocyclic compound used in the embodiments described herein.

The term “C₁-C₆” refers to 1 to 6 carbon atoms unless otherwise defined.

The term “C₃-C₈” refers to 3 to 8 carbon atoms unless otherwise defined. The term “C₁-C₆ alkyl” includes linear or branched alkyl groups such as methyl, ethyl, n-propyl, isopropyl, n-butyl, tert-butyl, sec-butyl, n-pentyl, and n-hexyl. The term “C₁-C₆ alkoxy” includes linear or branched alkoxy groups such as methoxy, ethoxy, n-propoxy, isopropoxy, n-butoxy, tert-butoxy, sec-butoxy, n-pentyloxy, and n-hexyloxy. The term “C₃-C₈ cycloalkyl” includes cyclopropyl, cyclobutyl, cyclopentyl, cyclohexyl, cydoheptyl, and cydooctyl. The term “halogen atom” includes fluorine, chlorine, bromine, and iodine.

In another embodiment of any of the methods of the present invention, the heterocyclic compound useful in the practice of the present invention selected from the group consisting of:
3,3-dimethylimidazo[1,2-a]pyridin-2(3H)-one,
3,3-dipropylimidazo[1,2-a]pyridin-2(3H)-one,
3,3-dibutylimidazo[1,2-a]pyridin-2(3H)-one,
3,3-diallylimidazo[1,2-a]pyridin-2(3H)-one,
3,3-diallyl-8-benzyloxyimidazo[1,2-a]pyridin-2(3H)-one,
3,3-di(2-propinyl)imidazo[1,2-a]pyridin-2(3H)-one,
3,3-dibenzylimidazo[1,2-a]pyridin-2(3H)-one,
3,3-dibenzyl-8-methylimidazo[1,2-a]pyridin-2(3H)-one,
3,3-dibenzyl-5,7-dimethylimidazo[1,2-a]pyridin-2(3H)-one,
3,3-dibenzyl-8-hydroxyimidazo[1,2-a]pyridin-2(3H)-one,
3,3-dibenzyl-8-methoxyimidazo[1,2-a]pyridin-2(3H)-one,
3,3-dibenzyl-8-ethoxyimidazo[1,2-a]pyridin-2(3H)-one,
8-allyloxy-3,3-dibenzylimidazo[1,2-a]pyridin-2(3H)-one,
3,3-dibenzyl-8-isopropoxyimidazo[1,2-a]pyridin-2(3H)-one,
3,3-dibenzyl-8-cyclopropylmethyloxyimidazo[1,2-a]pyridin-2(3H)-one,
3,3-dibenzyl-8-cycloheptyloxyimidazo[1,2-a]pyridin-2(3H)-one,
3,3-dibenzyl-6-chloroimidazo[1,2-a]pyridin-2(3H)-one,
3,3-dibenzyl-6,8-dichloroimidazo[1,2-a]pyridin-2(3H)-one,
3,3-dibenzyl-8-chloro-6-trifluoromethylimidazo[1,2-a]pyridin-2(3H)-one,
3,3-dibenzyl-8-benzyloxyimidazo[1,2-a]pyridin-2(3H)-one,
8-amino-3,3-dibenzylimidazo[1,2-a]pyridin-2(3H)-one,
8-acetylamino-3,3-dibenzylimidazo[1,2-a]pyridin-2(3H)-one,
3,3-dibenzyl-8-benzylaminoimidazo[1,2-a]pyridin-2(3H)-one,
3,3-bis(3-chlorobenzyl)imidazo[1,2-a]pyridin-2(3H)-one,
3,3-bis(3-fluorobenzyl)imidazo[1,2-a]pyridin-2(3H)-one,
3,3-bis(4-fluorobenzyl)imidazo[1,2-a]pyridin-2(3H)-one,
3,3-bis(2,4-dichlorobenzyl)imidazo[1,2-a]pyridin-2(3H)-one,
3,3-bis(4-dimethylaminobenzyl)imidazo[1,2-a]pyridin-2(3H)-one,
3,3-bis(4-methoxybenzyl)imidazo[1,2-a]pyridin-2(3H)-one,
3,3-bis(4-biphenylmethyl)imidazo[1,2-a]pyridin-2(3H)-one,
3,3-bis(4-cyanobenzyl)imidazo[1,2-a]pyridin-2(3H)-one,
3,3-bis(4-hydroxy-benzyl)imidazo[1,2-a]pyridin-2(3H)-one,
3,3-bis(3-phenyl-1-propyl)imidazo[1,2-a]pyridin-2(3H)-one,
3,3-bis(2,4-difluorobenzyl)imidazo[1,2-a]pyridin-2(3H)-one,
3,3-bis(4-nitrobenzyl)imidazo[1,2-a]pyridin-2(3H)-one,
3,3-bis(4-carboxybenzyl)imidazo[1,2-a]pyridin-2(3H)-one,
8-benzyloxy-3,3-bis(1-phenylethyl)imidazo[1,2-a]pyridin-2(3H)-one,
8-benzyloxy-3,3-bis(3-methylbenzyl)imidazo[1,2-a]pyridin-2(3H)-one,
8-benzyloxy-3,3-bis(4-methylbenzyl)imidazo[1,2-a]pyridin-2(3H)-one,
3-benzyl-3-(4-fluorobenzyl)imidazo[1,2-a]pyridin-2(3H)-one,
3-ethyl-3(4-fluorobenzyl)imidazo[1,2-a]pyridin-2(3H)-one,
8-methyl-3,3-bis(3-pyridylmethyl)imidazo[1,2-a]pyridin-2(3H)-one,
8-methyl-3,3-bis(4-pyridylmethyl)imidazo[1,2-a]pyridin-2(3H)-one,
3,3-bis(2-thienylmethyl)imidazo[1,2-a]pyridin-2(3H)-one,
3,3-bis(2-furylmethyl)imidazo[1,2-a]pyridin-2(3H)-one,
spiro(imidazo[1,2-a]pyridin-2(3H)-one-3,2′-indan),
spiro(imidazo[1,2-a]pyridin-2(3H)-one-3,2′-(2,3)dihydrophenarene),
spiro(imidazo[2,1-b]thiazol-6(5H)-one-5,2′-benzo(f)indan),
spiro(imidazo[1,2-b]thiazol-6(5H)-one-5,2′-indan),
spiro(2-methylimidazo[1,2-b]thiazol-6(5H)-one-5,2′-benzo(f)indan),
5,5-bis(4-fluorobenzyl)imidazo[2,1-b]thiazol-6(5H)-one,
5,5-dibenzylimidazo[2,1-b]thiazol-6(5H)-one,
5,5-bis(4-methylbenzyl)imidazo[2,1-b]thiazol-6(5H)-one,
5,5-bis(4-cyanobenzyl)imidazo[2,1-b]thiazol-6(5H)-one,
5,5-dibenzyl-2-methylimidazo[2,1-b]thiazol-6(5H)-one,
5,5-bis(4-fluorobenzyl)-2-methylimidazo[2,1-b]thiazol-6(5H)-one,
5,5-dicyclohexyl-2-methylimidazo[2,1-b]thiazol-6(5H)-one,
5,5-bis(4-cyanobenzyl)-2-methylimidazo[2,1-b]thiazol-6(5H)-one,
5,5-di(2-butenyl)imidazo[2,1-b]thiazol-6(5H)-one,
5,5-dibutylimidazo[2,1-b]thiazol-6(5H)-one,
5,5-dicyclohexylimidazo[2,1-b]thiazol-6(5H)-one,
5,5-bis(2-thienylmethyl)imidazo[2,1-b]thiazol-6(5H)-one,
spiro(2,3-dihydroimidazo[2,1-b]thiazol-6(5H)-one-5,2′-benzo(f)indan), 5,5-dibutyl-2,3-dihydroimidazo[2,1-b]thiazol-6(5H)-one,
5,5-di(2-butenyl)-2,3-dihydroimidazo[2,1-b]thiazol-6(5H)-one,
5,5-bis(4-methylbenzyl)-2,3-dihydroimidazo[2,1-b]thiazol-6(5H)-one,
5,5-bis(2-thienylmethyl)-2,3-dihydroimidazo[2,1-b]thiazol-6(5H)-one,
5,5-bis(4-fluorobenzyl)-2,3-dihydroimidazo[2,1-b]thiazol-6(5H)-one,
5,5-dibenzyl-2,3-dihydroimidazo[2,1-b]thiazol-6(5H)-one,
spiro(imidazo[1,2-a]pyridin-2(3H)-one-3,2′-benzo(f)indan),
2-hydroxy-3-(2-naphthylmethyl)-imidazo[1,2-a]pyridine,
3-benzylimidazo[1,2-a]pyridin-2(3H)-one,
spiro(5,6,7,8-tetrahydroimidazo[1,2-a]pyridin-2(3H)-one-3,2′-benzo(f)indan),
3,3-dicyclohexyl-5,6,7,8-tetrahydroimidazo[1,2-a]pyridin-2(3H)-one,
3,3-bis(2-thienylmethyl)-5,6,7,8-tetrahydroimidazo[1,2-a]pyridin-2(3H)-one,
3,3-dibutyl-5,6,7,8-tetrahydroimidazo[1,2-a]pyridin-2(3H)-one,
3,3-dipropyl-5,6,7,8-tetrahydroimidazo[1,2-a]pyridin-2(3H)-one,
spiro(imidazo[1,2-a]pyrimidin-2(3H)-one-3,2′-benzo(f)indan),
3,3-di(2-butenyl)imidazo[1,2-a]pyrimidin-2(3H)-one,
3,3-bis(2-thienylmethyl)imidazo[1,2-a]pyrimidin-2(3H)-one,
3,3-bis(4-fluorobenzyl)imidazo[1,2-a]pyrimidin-2(3H)-one,
3,3-dicyclohexylimidazo[1,2-a]pyrimidin-2(3H)-one,
3,3-bis(4-cyanobenzyl)imidazo[1,2-a]pyrimidin-2(3H)-one,
3,3-bis(4-methylbenzyl)imidazo[1,2-a]pyrimidin-2(3H)-one,
4,4-dibenzyl-1-methyl-5-oxo-4,5-dihydroimidazole,
spiro(imidazo[1,2-a]pyridin-2(3H)-one-3,2′-(4′-fluoroindan)),
spiro(imidazo[1,2-a]pyridin-2(3H)-one-3,2′-(5′-methoxyindan)),
spiro(imidazo[1,2-a]pyridin-2(3H)-one-3,2′-(5′-iodoindan)),
spiro(imidazo[1,2-a]pyridin-2(3H)-one-3,2′-(4′-cyanoindan)),
spiro(imidazo[2,1-a]isoquinolin-2(3H)-one-3,2′-indan),
spiro(imidazo[1,2-a]pyridin-2(3H)-one-3,2′-((1,2,5-thiadiazo)(4,5-c)indan)),
spiro(imidazo[2,1-a]isoquinolin-2(3H)-one-3 adiazo)(4,5-c)indan)),
spiro(imidazo[1,2-a]pyrimidin-2(3H)-one-3,4′-(1-cyclopentene)),
spiro(imidazo[1,2-a]pyrimidin-2(3H)-one-3,2′-indan),
spiro(imidazo[1,2-a]pyrimidin-2(3H)-one-3,1′-((1,2,5-thiadiazo)(4,5-c)indan)),
spiro(imidazo[1,2-a]pyridin-2(3H)-one-3,2′-(5′-trifluoromethylindan)),
spiro(imidazo(1,2-a)pyridin-2(3H)-one-3,2′-benzo(e)indan),
spiro(imidazo[2,1-a]isoquinolin-2(3H)-one-3,1′-(3′-cyclopentene)),
spiro(8-benzyloxyimidazo[1,2-a]pyridin-2(3H)-one-3,1′-(3′-cyclopentene)),
spiro(7,8,9,10-tetrahydroimidazo[2,1-a]isoquinolin-2(3H)-one-3,1′-cyclopentane),
spiro(imidazo[2,1-a]isoquinolin-2(3H)-one-3,1′-cyclopentane), and
spiro(5,6,7,8-tetrahydroimidazo(1,2-a)pyridin-2(3H)-one-3,2′-indan).

In another embodiment, the compound is spiro(imidazo[1,2-a]pyridin-2(3H)-one-3,2′-indan).

In another embodiment, the methods of the present invention can be practiced using any of the compounds disclosed in U.S. application Ser. No. 11/872,408 (published as US 2008/0103157 A1); U.S. application Ser. No. 11/872,418 (published as US 2008/0103158 A1); U.S. Pat. No. 6,635,652; U.S. Pat. No. 7,141,579; and international Appl. No. PCT/JP2007/070962 (published as WO 2008/047951), each of which is incorporated by reference in its entirety.

The compound ST101, also know as ZSET1446, has shown pharmacological activity in rodent models of learning and memory relevant to AD after both acute (single-dose) and chronic administration. The chemical name for ST101 is spiro(imidazo[1,2-a]pyridin-2(3H)-one-3,2′-indan).

For example, ST101 significantly improves age-impaired memory and attenuates memory deficits induced by chemical amnesic agents such as methamphetamine, the glutamate receptor antagonist, MK-801 and the muscarinic antagonist, scopolamine. (Yamaguchi Y., et al., J. Pharmacol. Exp. Ther. 317:1079-87 (2006); Ito Y., et al., J. Pharmacol. Exp. Ther. 320: 819-27 (2007)).

Experiments have shown that ST101 potentiates nicotine-stimulated release of acetylcholine (ACh), increases extracellular ACh concentrations in the cerebral cortex, and increases extracellular concentrations of both ACh and dopamine in the hippocampus. The breadth of models across which ST101 exerts its effects suggests the potential for involvement at an upstream target in the signaling pathway(s) associated with these processes.

ST101 has also demonstrated effects in the Senescence Accelerated Mouse 8 (SAMP8), a mouse strain that develops age-related deficits in learning and memory along with accumulation of Aβ-like deposits in brain tissue. The SAMP8 mouse is discussed in Morley, J. E., Biogerontology 3: 57-60 (2002). ST101 decreased accumulation of Aβ-like deposits and also produced an improvement in learning and memory functions, suggesting the behavioral effect of ST101 may be linked to reduction of Aβ production and/or deposition. See US 2008/103158 A1.

All patents, patent applications, and publications discussed herein are hereby incorporated by reference in their entireties.

### Example 1

### Effect of ST101 on β Amyloid in Vitro in N2a Cultured Cells

N2a is a murine neuroblastoma cell line that is known to produce amyloid peptides Aβ₁₋₄₀ and Aβ₁₋₄₂ in amounts measurable by ELISA assays. These forms of Aβ have been correlated with the pathology in AD brain and Aβ₁₋₄₂ in particular is postulated to have the ability to block α7 nicotinic receptors and to produce direct neurotoxic effects. N2a cells were treated for 24 hours with ST101 added to the tissue culture medium. Tissue culture medium was collected and analyzed by ELISA for the presence of Aβ.

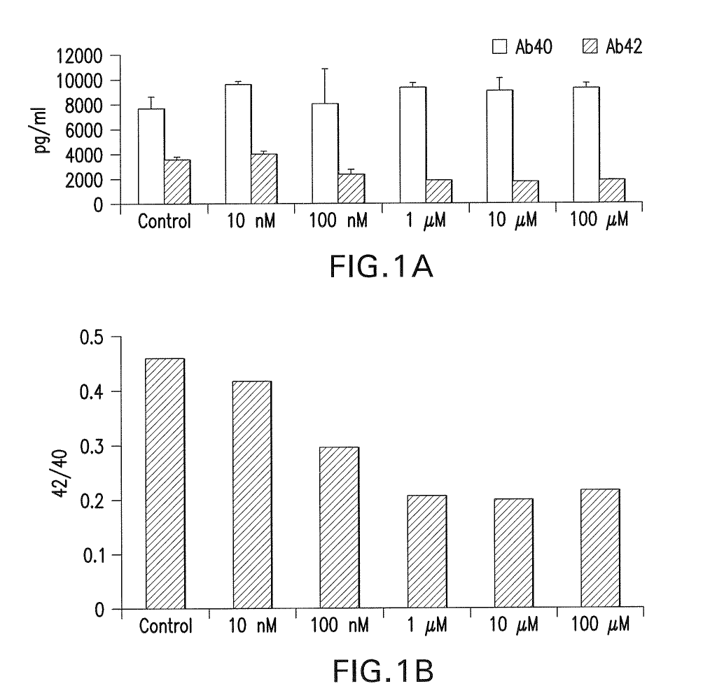

FIGS. 1A and 1B are bar graphs that depict the effect of the compound ST101 on Aβ production by N2a cells. FIG. 1A is a bar graph that depicts the Aβ concentration in the cell culture medium as a function of ST101 concentration compared to control. FIG. 1B a bar graph that depicts the ratio of Aβ₁₋₄₂ to Aβ₁₋₄₀ as a function of ST101 concentration compared to control. As shown in FIGS. 1A and 1B, ST101 significantly reduced Aβ₁₋₄₂ without major effects on Aβ₁₋₄₀ (FIG. 1).

### Example 2

### Effect of ST101 In 3xTg-AD Mice in the Morris Water Maze

Dr. Frank LaFerla's laboratory at the University of California, Irvine, has developed a transgenic mouse that contains 3 mutations relevant to Alzheimer's pathology (βAPPSwe, PS1M146V, and tauP301L) (Oddo et al., “Triple-transgenic model of AD with plaques and tangles: intracellular Aβ and synaptic dysfunction, Neuron 39(3):409-21 (2003)). These mutations shift APP cleavage from α- to β-secretase, increase production of Aβ₁₋₄₂ and drive the aggregation of tau into paired-helical filaments. The 3xTg-AD animals develop essential features of AD in an age-dependent fashion, with deficits in memory-related behavioral function, plaque and tangle pathology and synaptic dysfunction, including deficits in long-term potentiation, an activity believed critical to memory (Oddo et al., 2003). Furthermore, plaque formation precedes tangle formation and so mimics the development of the AD in humans. The 3xTg-AD mouse represents one of the closest animal models of AD developed to date.

ST101 Administration and Test Methods

3xTg-AD mice of approximately one year of age were treated for 2 months with ST101. An average dose of 5 mg/kg/day was administered in drinking water (calculated dose, based on mean water consumption). Behavioral effects were tested by assessing performance on the Morris Water Maze. Biochemical effects were examined by measuring brain content of Aβ and APP by ELISA and Western Blot.

Behavioral Effects: Performance of 3xTg-AD Mice in the Morris Water Maze (MWM), adapted from Roozendaal et al., Proc. Natl. Acad. Sci. U.S.A. 100: 1328-1333 (2003).

The MWM tests both spatial memory (i.e. hippocampus dependent) and cued learning (i.e. non-hippocampal) in rodents. The maze is a circular tank filled with opaque water. Mice are placed in the water and must swim to find and escape onto a platform submerged 1.5 cm beneath the surface of the water. The time (in seconds) required to find the platform is recorded. Animals rely on visual cues in the room containing the tank in order to find the platform on successive challenges. Training was conducted daily for seven consecutive days.

Retention of training was assessed twice, 24 and 72 hours after the final training trial. Animals were subjected to a 60-second free swim in the tank with the platform removed. Parameters measured included (1) latency: time required to reach the former platform location and (2) crosses: the number of times the animal swam across the former platform location. Decreases in latency and increases in crosses are indicative of improved spatial memory and cued learning.

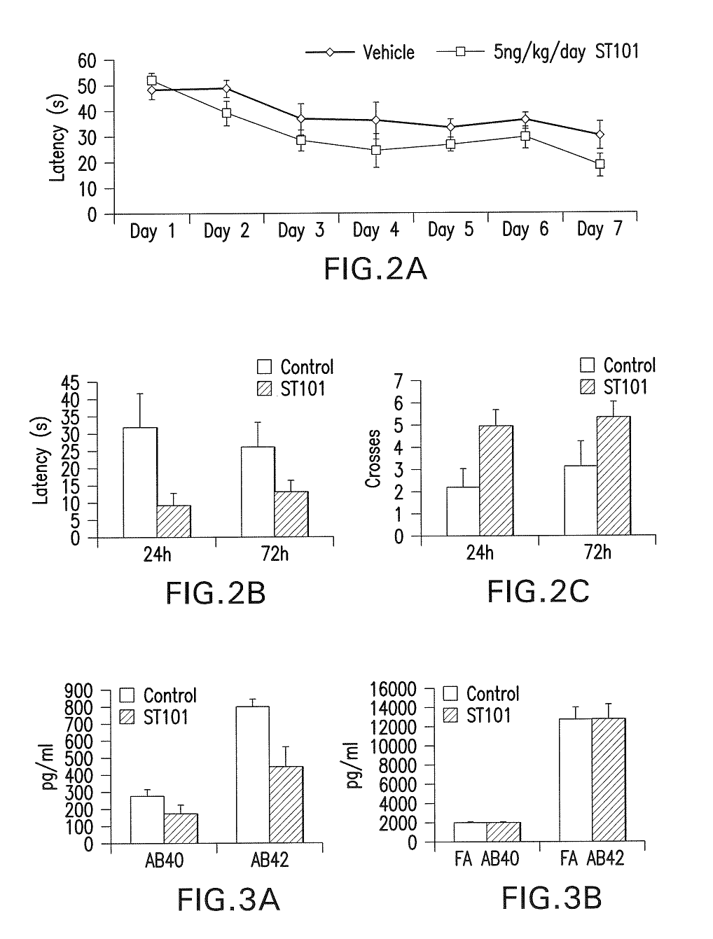

FIGS. 2A, 2B and 2C are graphs that depict the effect of ST101 in 3xTg-AD mice in the MWM. FIG. 2A is a line graph depicting latency (in seconds) during training, compared to control mice. FIGS. 2B and 2C are bar graphs that depict latency (in seconds) at 24 and 72 hours after training in ST101-treated animals and control mice.

As shown in FIG. 2A, ST101 and Control animals had similar latency on the first day of training. However, ST101-treated mice showed greater reductions in latency on successive days of the training compared with controls. FIGS. 2B and 2C also demonstrate both reductions in latency and increases in crosses during retention testing at both 24 and 72 hours. These data confirm that ST101 improves learning and memory performance in the 3xTg-AD mouse strain, which closely resembles human AD.

### Example 3

### Effect of ST101 on Aβ in Brain Tissue From 3xTg Mice-AD

Biochemical Effects: ST101 and Amyloid Processing Pathways

At the end of the 2-month treatment period, 3xTg Mice were sacrificed and brain tissue was processed. In the first set of analyses, soluble Aβ₁₋₄₀ and Aβ₁₋₄₂, as well as insoluble Aβ (after formic acid extraction), were quantified by ELISA. Soluble Aβ represents protein that has been processed from full length APP and released. Insoluble Aβ represents fibrillar aggregates that are ultimately deposited in amyloid plaques.

FIGS. 3A and 3B are bar graphs that depict the effect of ST101 on Aβ in brain tissue from 3xTg mice-AD. FIG. 3A depicts the amounts of soluble Aβ₁₋₄₀ and Aβ₁₋₄₂ in brain tissue in mice treated with ST101, relative to control mice. FIG. 3B a bar graph that depicts the amounts of insoluble Aβ₁₋₄₀ and Aβ₁₋₄₂ (formic acid extraction) in mice treated with ST101, relative to control mice. One animal in the ST101 treated group in panel A was excluded due to analytical artifact.

As shown in FIGS. 3A and 3B, ST101-treated mice had significantly decreased levels of soluble Aβ₁₋₄₂ and moderately decreased soluble Aβ₁₋₄₀. Insoluble Aβ was unaffected. These results suggest that ST101 may impact Aβ production or release, but that it has no measurable effect on Aβ that has already formed insoluble aggregates.

### Example 4

### APP C-Terminal Fragments Detected by Antibody CT20

To attempt to determine at what part in the Aβ processing/release pathway ST101 may be active, a series of Western blot analyses of brain extracts from the same mice were conducted. These Westerns blots examined intact APP as well as products of its post-translational processing and subsequent degradation.

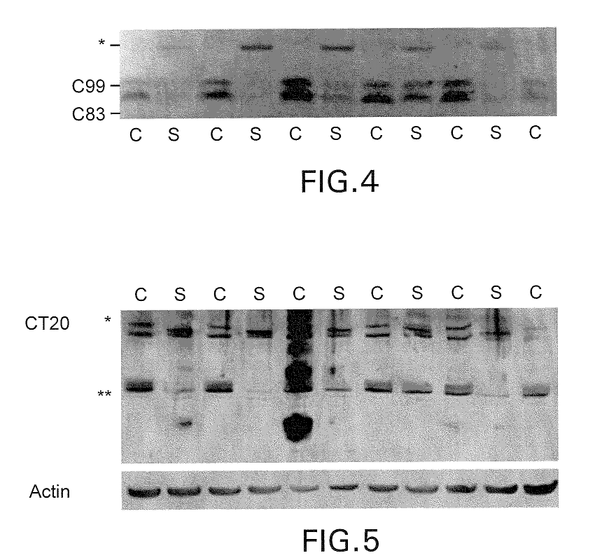

FIG. 4 is a Western blot that depicts APP C-terminal fragments detected by antibody CT20 in the brains of ST101-treated (S) 3xTg-AD mice, relative to untreated (C) 3xTg mice-AD.

As shown in FIG. 4, antibody CT20 (directed against the C-terminus of APP) revealed a substantial decrease in C99 and C83 C-terminal APP fragments. These fragments are byproducts of (3-secretase and a-secretase cleavage, respectively. Also shown is the appearance of a novel, longer C-terminal fragment of about 17 kDa molecular weight (indicated by *).

### Example 5

### APP and Degradation Fragment Detected by Antibody CT20

FIG. 5 is a Western blot that depicts APP and degradation fragments detected by antibody CT20 in the brains of ST101-treated (S) 3xTg-AD mice, relative to untreated (C) 3xTg-AD mice. “CT20” stands for full length APP species, and “Actin” stands for anti-beta-actin antibody as a protein loading control.

The Western blot analysis detected full-length unprocessed APP in all extracts (FIG. 5,*). Subtle band shifts suggested additional ST101-induced modification of APP, e.g., slightly lowered molecular weight of some full-length species (possible change in glycosylation, phosphorylation or other post-translational modifications) and the disappearance or significant reduction of a major APP degradation intermediate (˜50 kDa) (FIG. 5, **).

### Example 6

### Alternative Amyloid Processing Pathway

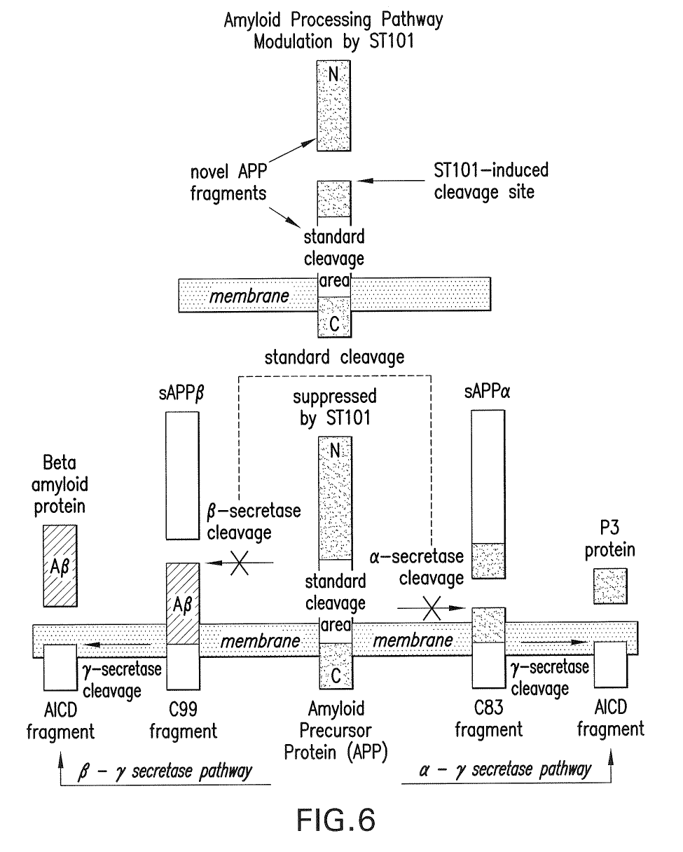

FIG. 6 is a drawing that depicts a proposed amyloid processing pathway leading to a novel APP C-terminal fragment. The proposed pathway explains the appearance of the novel approximately 17 kD fragment shown in the Western blot from FIG. 4. This fragment is generated by cleavage at an uncharacterized site about 60 amino acids N-terminal to the β-secretase cleavage site.

The new pathway appears to preempt both α- and β-secretase cleavage, as the usual products of these cleavage events are greatly reduced (C83 and C99 for α- and β-secretase, respectively), and therefore the cleavage sites that are the targets for these enzymes remain intact.

This alteration of APP metabolism induced by ST101 is accompanied by marked improvement in learning and memory tasks in an animal model arguably considered to be a close representation of clinical AD. When viewed in conjunction with earlier non-clinical data, it appears ST101 may operate at physiological processes upstream of those of both marketed agents and agents currently under investigation with known mechanisms of action and thus, represents a new avenue of treatment for AD.

### Example 7

### APP C-Terminal Fragments Detected by Antibody CT20

3xTg-AD mice of approximately 3 months of age were treated for 10 months with ST101. An average dose of 5 or 1 or 0.1 mg/kg/day was administered in drinking water (calculated dose, based on mean water consumption).

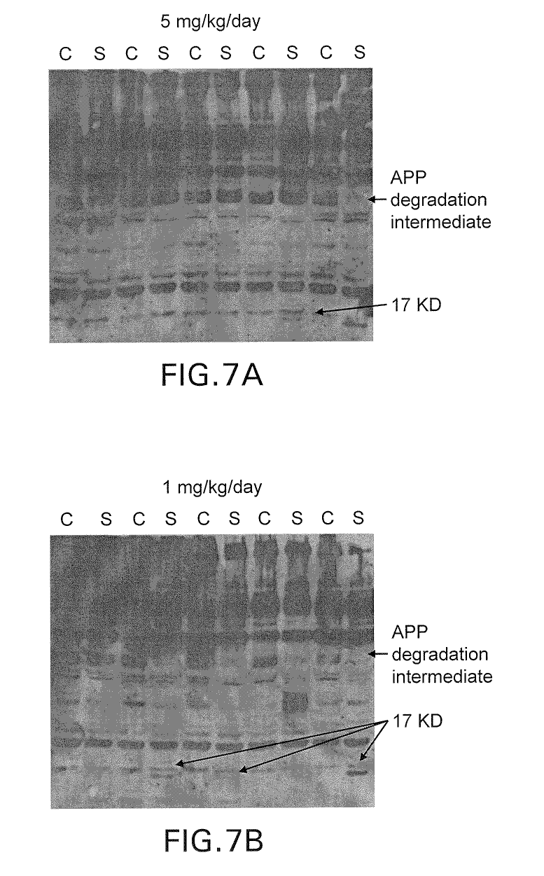

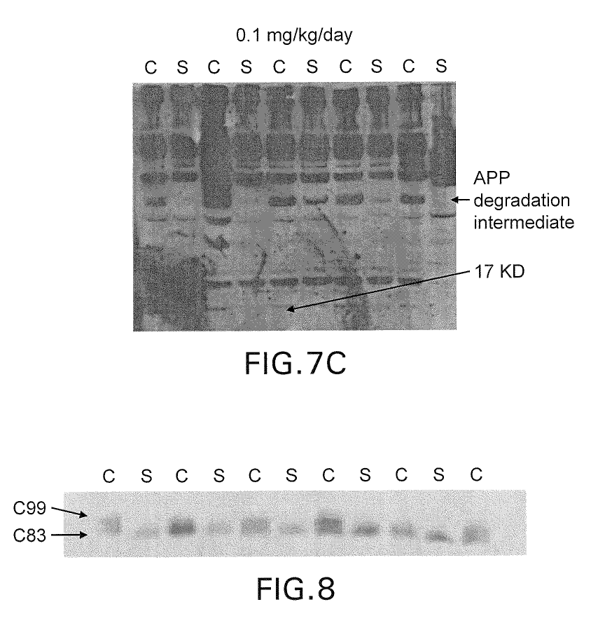

FIGS. 7A-C are Western blots that depict APP C-terminal fragments detected by antibody CT20 in the brains of ST101-treated (S) 3xTg-AD mice, relative to untreated (C) 3xTg-AD mice.

As shown in FIGS. 7A-7C, antibody CT20 (directed against the C-terminus of APP) revealed the disappearance or significant reduction of a major APP degradation intermediate (˜50 kDa) in some samples similar to the effect seen in FIG. 5, **. Also shown is the appearance of a novel, longer C-terminal fragment of about 17 kDa molecular weight in some samples similar to the effect seen in FIG. 4.

### Example 8

### APP C-Terminal Fragments Detected by Antibody 1565-1

3xTg-AD mice of approximately 12 months of age were treated for 2.5 months with ST101. An average dose of 5 mg/kg/day was administered in drinking water (calculated dose, based on mean water consumption).

FIG. 8 is a Western blot that depicts C-terminal fragments detected by antibody 1565-1 (Epitomics Inc.) in the brains of ST101-treated (S) 3xTg-AD mice, relative to untreated (C) 3xTg mice-AD.

As shown in FIG. 8, antibody 1565-1 (directed against a peptide near the C-terminus of APP) revealed the disappearance or significant reduction of the APP C99 fragment. A longer C-terminal fragment of about 17 kDa molecular weight was not observed.

The breadth and scope of the present invention should not be limited by any of the above-described exemplary embodiments, but should be defined only in accordance with the following claims and their equivalents.

What is claimed is:

1. A method of inducing cleavage of amyloid precursor protein to produce an approximately 17 kilodalton carboxy-terminal fragment of amyloid precursor protein in a subject, the method comprising administering a heterocyclic compound having the general Formula (I): 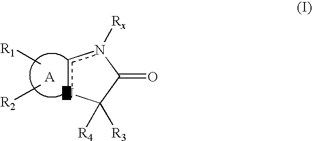
or a pharmaceutically acceptable salt, hydrate or prodrug thereof to a subject in need thereof,
wherein the approximately 17 kilodalton fragment includes the carboxyterminal amino acid sequence of amyloid precursor protein and amyloid-beta amino acid sequence, and wherein each of Rₓ, R₁, R₂, R₃, R₄ are as defined herein.

2. The method of claim 1, wherein the heterocyclic compound is spiro(imidazo[1,2-a]pyridin-2(3H)-one-3,2′-indan).

3. The method of claim 1, wherein administering said heterocyclic compound results in a decrease in the production of one or more of Aβ₁₋₄₂, Aβ₁₋₄₀, the C99 fragment of amyloid precursor protein, or the C83 fragment of amyloid precursor protein.

4. The method of claim 1, wherein the subject has Alzheimer's Disease.

5. The method of claim 4, wherein the subject has been diagnosed with Alzheimer's Disease.

6. An isolated approximately 17 kilodalton amyloid precursor protein fragment that includes the carboxyterminal amino acid sequence of amyloid precursor protein and amyloid-beta amino acid sequence.

7. A composition comprising the fragment of claim 6.

8. A method for screening for a compound that cleaves amyloid precursor protein to generate an approximately 17 kilodalton fragment of amyloid precursor protein, said method comprising:
(a) exposing cells that produce amyloid precursor protein or fragments thereof to a test compound, and
(b) detecting the amount of the approximately 17 kilodalton fragment,
wherein the approximately 17 kilodalton fragment, includes the carboxyterminal amino acid sequence of amyloid precursor protein and amyloid-beta amino acid sequence, and
wherein an increase in the amount of the approximately 17 kilodalton fragment in cells exposed to the compound, relative to the amount of the approximately 17 kilodalton fragment in cells that are not exposed to the compound, indicates that the compound induces cleavage of amyloid precursor protein to generate the approximately 17 kilodalton fragment.

9. The method of claim 8, further comprising (c) determining whether the amount of one or more of Aβ₁₋₄₂, Aβ₁₋₄₀, the C99 fragment of amyloid precursor protein, or the C83 fragment of amyloid precursor protein in cells exposed to the compound is decreased, relative to the amount of Aβ₁₋₄₂, Aβ₁₋₄₀, the C99 fragment of amyloid precursor protein, or the C83 fragment of amyloid precursor protein in cells that are not exposed to the compound.

10. The method of claim 1, wherein said screening method is carried out in vitro.

11. The method of claim 8, further comprising (c) determining whether the amount of one or more of Aβ₁₋₄₂, Aβ₁₋₄₀, the C99 fragment of amyloid precursor protein, or the C83 fragment of amyloid precursor protein in the cell culture lysate of cells exposed to the compound is decreased, relative to the amount of Aβ₁₋₄₂, Aβ₁₋₄₀, the C99 fragment of amyloid precursor protein, or the C83 fragment of amyloid precursor protein in the cell culture medium of cells that are not exposed to the compound.

12. The method of claim 8, wherein said screening method is carried out in a high-throughput manner.

13. A method for screening for a compound that cleaves amyloid precursor protein to generate an approximately 17 kilodalton fragment of APP, the method comprising:
(a) exposing cells that produce amyloid precursor protein or fragments thereof to a test compound, and
(b) detecting the approximately 17 kilodalton fragment,
wherein the approximately 17 kilodalton fragment includes the carboxy-terminal amino acid sequence of amyloid precursor protein and amyloid-beta amino acid sequence, and
wherein the presence of the approximately 17 kilodalton fragment of cells exposed to the compound, relative to the absence of the approximately 17 kilodalton fragment in cells that are not exposed to the compound, indicates that the compound induces cleavage of amyloid precursor protein to generate the approximately 17 kilodalton fragment.

14. The method of claim 13, further comprising (c) determining whether the amount of one or more of Aβ₁₋₄₂, Aβ₁₋₄₀, the C99 fragment of amyloid precursor protein, or the C83 fragment of amyloid precursor protein in cells exposed to the compound is decreased, relative to the amount of Aβ₁₋₄₂, Aβ₁₋₄₀, the C99 fragment of amyloid precursor protein, or the C83 fragment of amyloid precursor protein in cells that are not exposed to the compound.

15. The method of claim 13, wherein said screening method is carried out in vitro.

16. The method of claim 13, further comprising (c) determining whether the amount of one or more of Aβ₁₋₄₂, Aβ₁₋₄₀, the C99 fragment of amyloid precursor protein, or the C83 fragment of amyloid precursor protein in the cell culture lysate of cells exposed to the compound is decreased, relative to the amount of Aβ₁₋₄₂, Aβ₁₋₄₀, the C99 fragment of amyloid precursor protein, or the C83 fragment of amyloid precursor protein in the cell culture medium of cells that are not exposed to the compound.

17. The method of claim 13, wherein said screening method is carried out in a high-throughput manner.

18. A method of inducing cleavage of amyloid precursor protein to produce an approximately 17 kilodalton carboxy-terminal fragment of amyloid precursor protein in a subject, the method comprising administering a compound that is not a compound having the general Formula (I): 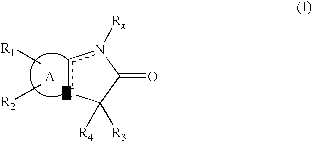
or a pharmaceutically acceptable salt, hydrate or prodrug thereof,
wherein each of Rₓ, R₁, R₂, R₃, R₄ are as defined herein.

19. The method of claim 18, wherein administering said heterocyclic compound results in a decrease in the production of one or more of Aβ₁₋₄₂, Aβ₁₋₄₀, the C99 fragment of amyloid precursor protein, or the C83 fragment of amyloid precursor protein.

20. The method of claim 1, wherein the subject has Alzheimer's Disease.

21. The method of claim 20, wherein the subject has been diagnosed with Alzheimer's Disease.
